(19) United States
(12) Patent Application Publication
(10) Pub. No.: US 2023/0355598 A1
(43) Pub. Date: Nov. 9, 2023

(54) PYRROLIDINE COMPOUNDS TO TREAT XERODERMA PIGMENTOSUM

(71) Applicant: Clinuvel (UK) Ltd., London (GB)

(72) Inventor: Philippe WOLGEN, London (GB)

(21) Appl. No.: 18/024,572

(22) PCT Filed: Sep. 9, 2021
(86) PCT No.: PCT/EP2021/074844

§ 371 Date: Mar. 3, 2023

Related U.S. Application Data
(60) Provisional application No. 63/076,161, filed on Sep. 9, 2020.

(30) Foreign Application Priority Data
Sep. 9, 2020 (EP) 63/076161

(51) Int. Cl. A61K 31/454

(52) U.S. Cl. CPC A61K 31/454

(57) ABSTRACT

The present invention relates to pyrrolidine compounds for treatment of Xeroderma Pigmentosum (XP), specifically for enhancing DNA repair in a subject suffering from XP.

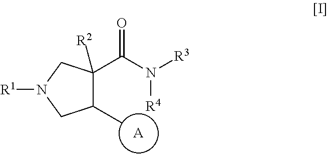

### TECHNICAL FIELD

The present invention is directed to compounds for use in treatment of a human subject suffering from a particular medical indication, to a composition, to a method of treating a medical indication, and to the manufacture of a medicament for the treatment of a medical indication.

### BACKGROUND TO THE INVENTION

Xeroderma Pigmentosum (XP) is an autosomal recessive genetic disorder in which repair of DNA (deoxyribonucleic acid) is deficient. There is a clinical need for improvements of treatment of XP and of reducing its symptoms and improving the quality of life of XP subjects.

### SUMMARY OF THE INVENTION

According to the invention, we have found surprising benefits of pyrrolidine compounds in treatment of Xeroderma Pigmentosum. Accordingly, the present invention relates to a pyrrolidine compound of the invention for use in the treatment of a human subject suffering from Xeroderma Pigmentosum (XP). Specifically, the present invention is directed to use in enhancing photo (light)-induced DNA repair, in particular Ultra Violet (UV)-induced DNA repair.

In one embodiment of the invention, the pyrrolidine compound is represented by formula [I]:

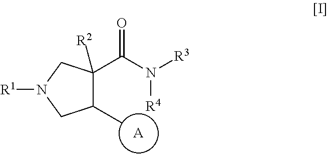

wherein
ring A represents an optionally substituted aryl group or an optionally substituted heteroaryl group;
R¹ represents an optionally substituted alkyl group, an optionally substituted cycloalkyl group, an optionally substituted aliphatic heterocyclic group, an optionally substituted aryl group that may be partially hydrogenated, an optionally substituted heteroaryl group, or an optionally substituted carbamoyl group;
R² represents a halogen atom, an alkyl group, or an optionally substituted alkoxy group;
R³ is an alkyl group substituted with an optionally substituted aryl group, or an alkyl group substituted with an optionally substituted heteroaryl group and R⁴ is a hydrogen atom or an alkyl group; or R³ and R⁴ are terminally attached to each other, and together with the nitrogen atom to which they are attached, form an optionally substituted nitrogen-containing aliphatic heterocyclic ring that may partially contain a double bond;
or a pharmaceutically acceptable salt thereof.

In a specific embodiment, the invention is directed to 1-{2-[(3S,4R)-1-{[(3R,4R)-1-cyclopentyl-3-fluoro-4-(4-methoxyphenyl)pyrrolidin-3-yl]carbonyl}-4-(methoxymethyl)pyrrolidin-3-yl]-5-(trifluoromethyl)phenyl}piperidine-4-carboxylic acid or a pharmaceutically acceptable salt thereof.

Preferably, the compound for use according to the invention is a 3,3-di-substituted pyrrolidine compounds with 2 substituents in the 3-position of pyrrolidine and wherein the compound preferably has substituents in the 1-, 3-, and 4-positions.

Preferably, the compound is administered orally. Preferably, the compound is administered daily. Preferably, the compound is administered at least 3 times consecutively to the human subject.

Preferably, the compound is used for treatment of XP selected from complementation group A (XP-A), complementation group B (XP-B), complementation group C (XP-C), complementation group D (XP-D), complementation group E (XP-E), complementation group F (XP-F), complementation group G (XP-G), and variant type (XP-V), more preferably treatment of complementation group C (XP-C).

The invention further relates to a method of treating Xeroderma Pigmentosum (XP) by administering a pyrrolidine compound of the invention, preferably to enhance DNA repair in the human subject suffering from XP.

The invention further relates to use of a pyrrolidine compound for the manufacture of a medicament for the treatment of Xeroderma Pigmentosum, preferably by enhancing DNA repair.

In a further embodiment of the present invention, the invention provides for effective yet safe and convenient treatment of XP by using a pyrrolidine compound of the invention, preferably at least partially reducing one or more of the symptoms associated with XP, and, specifically, the invention is directed to enhancing UV-induced DNA repair.

In a further embodiment, we have surprisingly found that the pyrrolidine compounds of the invention can be beneficially used in the treatment of human subjects suffering from chronic photo damage of the skin, particularly due to UV light. This applies especially to human subjects suffering from poikiloderma. This particularly applies to human subjects suffering from XP. In fact, generally MC1R agonists can be beneficially used for these purposes. This includes MC1R agonists afamelanotide and the other MC1R agonist compounds referred to in U.S. Patent Publication No. 2020/0246436 (U.S. patent application Ser. No. 16/482,614), the contents of which are herein incorporated by reference. Without wishing to be bound by any theory, it is believed that leathery skin due to photo damage of the skin can be improved, leading to less hyperkeratosis, improving the skin architecture, and restoring normal skin proportions (for instance, less cell mitosis, better basal membrane, more stroma, and/or more fluid in and around the cell). Investigating skin samples under the microscope, pathologists use a rating system for diagnosis of loss of skin architecture (tissue dysplastic). Accordingly, the present invention relates to pyrrolidine compounds of the invention for use in the treatment of chronic photo damage, particularly in XP patients. The present invention extends to MC1R agonist compounds such as afamelanotide and the other compounds mentioned in U.S. Patent Publication No. US2020/0246436 which can be used for that same purpose.

### DETAILED DESCRIPTION OF THE INVENTION

For the purpose of this invention, treatment is defined as encompassing prevention of a disorder. Further, treatment is defined as encompassing reduction of symptoms associated with the disease.

For the purpose of this invention, the pyrrolidine compounds of the invention may be used as such or in the form of a pharmaceutically acceptable salt thereof. Examples of pharmaceutically acceptable salts include inorganic acid salts such as hydrochloride, sulfate, phosphate, and hydrobromate; and organic acid salts such as acetate, fumarate, oxalate, citrate, methanesulfonate, benzenesulfonate, tosylate, and maleate. Preferred examples of such salts are acetate, trifluoroacetate, sulfate, and chloride salts. The acetate salt is generally most preferred.

According to the invention, we have surprisingly found that pyrrolidine compounds of the invention are effective in treatment of human subjects suffering from Xeroderma Pigmentosum (XP). For the purpose of this invention, the term Xeroderma Pigmentosum refers to an autosomal recessive, genetic disorder of human subjects characterized by an extreme sensitivity to UV rays. Symptoms of XP usually occur at infancy or early childhood and include freckling of the sun-exposed skin, dry skin, and changes in pigmentation. Subjects suffering from XP are particularly susceptible to increased risk of various cancer types and neurological abnormalities. Without wishing to be bound by any theory, it is believed that the ability to repair DNA damage in XP subjects is compromised and/or at least partially disabled.

At least eight inherited forms of XP have been identified based on genes, including complementation group A (XP-A), complementation group B (XP-B), complementation group C (XP-C), complementation group D (XP-D), complementation group E (XP-E), complementation group F (XP-F), complementation group G (XP-G), and variant type (XP-V).

Accordingly, the invention relates to treatment of complementation group A (XP-A). The invention further relates to treatment of complementation group B (XP-B). The invention further relates to treatment of complementation group C (XP-C) which is a particularly preferred group, generally involving the highest rate of DNA damage and of skin cancer. The invention further relates to treatment of complementation group D (XP-D). The invention further relates to treatment of complementation group E (XP-E). The invention further relates to treatment of complementation group F (XP-F). The invention further relates to treatment of complementation group G (XP-G). And the invention further relates to treatment of variant type (XP-V). In particularly, the invention is preferably directed to treatment of complementation group A (XP-A), complementation group B (XP-B), complementation group C (XP-C) and complementation group F (XP-F). Especially preferred is complementation group C (XP-C) in view of the highest rate of DNA damage.

The present invention relates to the use of a pyrrolidine compound, wherein the compound preferably has agonist MC1R activity. Different pyrrolidine compounds have been described in the art and have been proposed for various purposes. For instance, U.S. Patent Publication No. 2017/190697 discloses pyrrolidine compounds and discusses their synthesis and their potential use in various medical indications such as pigmentation. Importantly, the compounds described in U.S. Patent Publication No. US2017/190697 and their synthesis are incorporated in the present application by reference.

It is preferred according to the present invention to administer the pyrrolidine compound of the present invention systemically. Preferably, the pyrrolidine compound is administered orally or transdermally or cutaneously, and more preferably orally. One systemic administration of the pyrrolidine compound of the invention is by way of oral administration, preferably in a controlled release composition.

Preferably, exposure of the XP subject to the pyrrolidine compound of the composition of the invention is for at least 1 day and preferably for instance up to 100 days. According to a preferred treatment of the invention, the pyrrolidine compound is administered at least 2 times to the subject, more preferably at least 3 times, most preferably at least 5 times and for instance up to 100 times, more preferably up to 20 times, each composition providing the above mentioned exposure. Preferably, the dosing is consecutively. Dosing of the pyrrolidine compound is preferably daily and preferably orally. As will be understood by a skilled person in the art, after initial release of the pyrrolidine compound from the drug composition and absorption by the subject into the blood plasma, the pyrrolidine compound will be present in the blood plasma of the subject at the level and the time period indicated. Subsequently, the next dose is administered. Dose levels, plasma levels and dose frequencies may vary and are each preferably—independently—within the ranges given above. Thus, the pyrrolidine compound is administered in a composition and in an amount that results in the blood plasma levels indicated. Accordingly, the human subject is subjected to the blood plasma levels indicated. It will be understood that for the purpose of the invention, intervals are separate and subsequent and do not overlap.

According to one aspect, the invention is directed to pyrrolidine compounds. The term “pyrrolidine compound” as used herein is defined as a compound with formula (I)

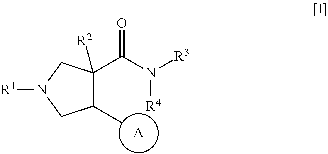

wherein:
ring A represents an optionally substituted aryl group or an optionally substituted heteroaryl group;
R¹ represents an optionally substituted alkyl group, an optionally substituted cycloalkyl group, an optionally substituted aliphatic heterocyclic group, an optionally substituted aryl group that may be partially hydrogenated, an optionally substituted heteroaryl group, or an optionally substituted carbamoyl group;
R² represents a halogen atom, an alkyl group, or an optionally substituted alkoxy group;
R³ is an alkyl group substituted with an optionally substituted aryl group, or an alkyl group substituted with an optionally substituted heteroaryl group and R⁴ is a hydrogen atom or an alkyl group; or R³ and R⁴ are terminally attached to each other, and together with the nitrogen atom to which they are attached, form an optionally substituted nitrogen-containing aliphatic heterocyclic ring that may partially contain a double bond;
or a pharmaceutically acceptable salt thereof.

Preferably, compounds represented by below formulas (a) (b), (c), (d), and (e) are excluded (Formulae a, b, c, d, and e):

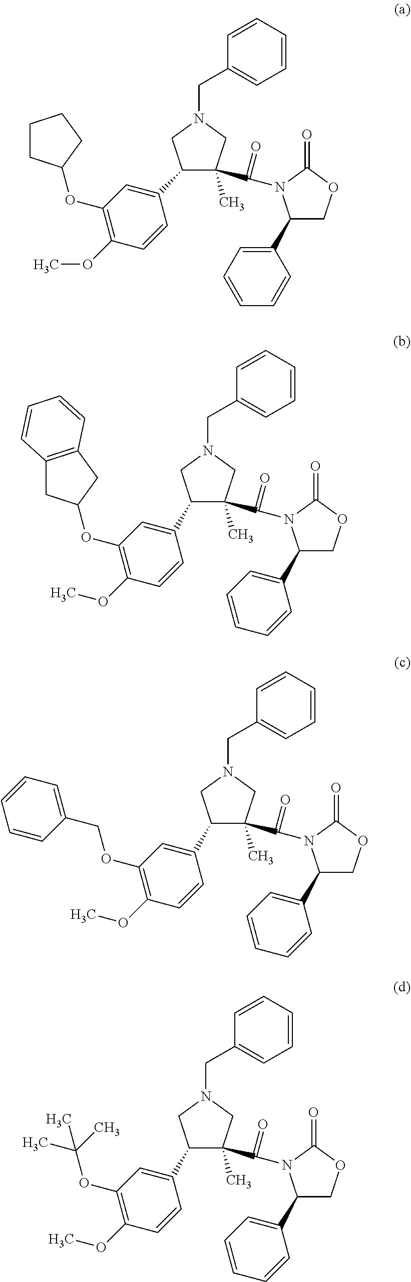

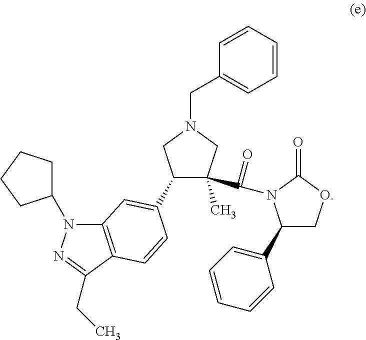 [SMILES: CCc1nn(C2CCCC2)c2cc([C@@H]3CN(Cc4ccccc4)C[C@@]3(C)C(=O)N3C(=O)OC[C@H]3c3ccccc3)ccc12]

Preferably, the pyrrolidine compound exhibits agonist activity for the melanocortin-1-receptor (MC1R), the receptor to which alpha-MSH binds to initiate the production of melanin within a melanocyte.

Preferably, the invention is directed to a pyrrolidine compound or pharmaceutically acceptable salt thereof of formula (I), wherein:
ring A is an optionally substituted aryl group or an optionally substituted heteroaryl group, wherein substituent(s) on each of the optionally substituted aryl group and the optionally substituted heteroaryl group is/are one to three group(s) independently selected from the group consisting of a halogen atom, an alkyl group, a haloalkyl group, a cycloalkyl group, an alkoxy group, a haloalkoxy group, and an alkyleneoxy group;
R¹ is an optionally substituted alkyl group, an optionally substituted cycloalkyl group, an optionally substituted aliphatic heterocyclic group, an optionally substituted aryl group that may be partially hydrogenated, an optionally substituted heteroaryl group, or a carbamoyl group optionally substituted with one or two alkyl group(s), wherein substituent(s) on the optionally substituted alkyl group is/are one to three group(s) independently selected from the group consisting of a halogen atom; a hydroxy group; an oxo group; a cyano group; a cycloalkyl group; an alkoxy group; an alkanoyl group; a carbamoyl group optionally substituted with one or two alkyl group(s); an aliphatic heterocyclic group; an aliphatic heterocyclic carbonyl group optionally substituted with one or two group(s) independently selected from the group consisting of a halogen atom, an alkyl group, a haloalkyl group, and an alkoxyalkyl group; an alkylsulfonyl group; an aliphatic heterocyclic sulfonyl group; and an alkyleneoxy group, and substituent(s) on each of the optionally substituted cycloalkyl group, the optionally substituted aliphatic heterocyclic group, the optionally substituted aryl group that may be partially hydrogenated, and the optionally substituted heteroaryl group is/are one to three group(s) independently selected from the group consisting of a halogen atom; a hydroxy group; an oxo group; a cyano group; an alkyl group; a haloalkyl group; a cycloalkyl group; an alkoxy group; a hydroxyalkyl group; an alkoxyalkyl group; an alkanoyl group; a carbamoyl group optionally substituted with one or two alkyl group(s); an aliphatic heterocyclic group; an aliphatic heterocyclic carbonyl group optionally substituted with one or two group(s) independently selected from the group consisting of a halogen atom, an alkyl group, a haloalkyl group, and an alkoxyalkyl group; an alkylsulfonyl group; an aliphatic heterocyclic sulfonyl group; and an alkyleneoxy group;
R² is a halogen atom, an alkyl group, or an alkoxy group;
R³ is an alkyl group substituted with a substituted aryl group, or an alkyl group substituted with a substituted heteroaryl group, wherein a substituent on each of the substituted aryl group and the substituted heteroaryl group is an aliphatic heterocyclic group optionally substituted with a carboxyl group, and the aryl group and the heteroaryl group are each optionally further substituted with a haloalkyl group; and
R⁴ is a hydrogen atom or an alkyl group; or
R³ and R⁴ are terminally attached to each other, and together with the nitrogen atom to which they are attached, form a group represented by formula [II]:

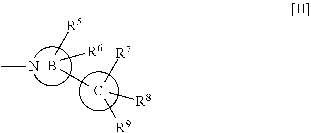

wherein:
ring B represents a nitrogen-containing aliphatic heterocyclic group that may partially contain a double bond;
ring C represents an aryl group or a heteroaryl group;
R⁵ and R⁶ each independently represent a group selected from the group consisting of a hydrogen atom, a halogen atom, a cyano group, an alkyl group, a haloalkyl group, a cyanoalkyl group, a hydroxyalkyl group, an alkoxyalkyl group, a carboxyl group, a carbamoyl group optionally substituted with one or two alkyl group(s), and an alkoxy group;
R⁷ represents an optionally substituted alkyl group, an optionally substituted alkenyl group, an optionally substituted cycloalkyl group, an optionally substituted cycloalkenyl group, an optionally substituted aryl group, an optionally substituted heteroaryl group, an optionally substituted aliphatic heterocyclic group, an optionally substituted alkoxy group, an amino group optionally substituted with one or two alkyl group(s) optionally substituted with a carboxyl group, or a carbamoyl group optionally substituted with one or two alkyl group(s) optionally substituted with a carboxyl group,
wherein substituent(s) on the optionally substituted alkyl group is/are one or two group(s) independently selected from the group consisting of a hydroxy group; an oxo group; a cyano group; an alkoxy group; an alkanoyl group; a carboxyl group; an alkoxycarbonyl group; an aliphatic heterocyclic carbonyl group optionally substituted with a carboxyl group; a heteroaryl group optionally substituted with a hydroxy or an oxo group; an aliphatic heterocyclic group optionally substituted with one or two oxo group(s); a carbamoyl group optionally substituted with one or two group(s) independently selected from the group consisting of an alkyl group (the alkyl moiety is optionally substituted with a hydroxyl group, an alkoxy group, or a carboxyl group) and a hydroxy group; an alkylsulfonyl group; an aminosulfonyl group optionally substituted with one or two alkyl group(s); an aminosulfonylaminocarbonyl group optionally substituted with one or two alkyl group(s); an alkylsulfonylaminocarbonyl group; and an amino group optionally substituted with one or two group(s) independently selected from the group consisting of an alkyl group, an alkanoyl group, and an alkylsulfonyl group, and substituent(s) on each of the optionally substituted alkenyl group, the optionally substituted cycloalkyl group, the optionally substituted cycloalkenyl group, the optionally substituted aryl group, the optionally substituted heteroaryl group, the optionally substituted aliphatic heterocyclic group, and the optionally substituted alkoxy group is/are one or two group(s) independently selected from the group consisting of a hydroxy group; an oxo group; a cyano group; an alkyl group optionally substituted with a carboxyl group; an alkoxy group; an alkanoyl group; a carboxyl group; an alkoxycarbonyl group; an aliphatic heterocyclic carbonyl group optionally substituted with a carboxyl group; a heteroaryl group optionally substituted with a hydroxy or an oxo group; an aliphatic heterocyclic group optionally substituted with one or two oxo group(s); a carbamoyl group optionally substituted with one or two group(s) independently selected from the group consisting of an alkyl group (the alkyl moiety is optionally substituted with a hydroxy group, an alkoxy group, or a carboxyl group) and a hydroxy group; an alkylsulfonyl group; an aminosulfonyl group optionally substituted with one or two alkyl group(s); an aminosulfonylaminocarbonyl group optionally substituted with one or two alkyl group(s); an alkylsulfonylaminocarbonyl group; and an amino group optionally substituted with one or two group(s) independently selected from the group consisting of an alkyl group, an alkanoyl group, and an alkylsulfonyl group; and
R⁸ and R⁹ each independently represent a group selected from the group consisting of a hydrogen atom, a halogen atom, a cyano group, an alkyl group, a haloalkyl group, and a haloalkoxy group.

Preferably, the invention is directed to a pyrrolidine compound or pharmaceutically acceptable salt thereof of above-mentioned formula (I), wherein:
ring A is an optionally substituted aryl group or an optionally substituted heteroaryl group, wherein substituent(s) on each of the optionally substituted aryl group and the optionally substituted heteroaryl group is/are one to three group(s) independently selected from the group consisting of a halogen atom, an alkyl group, a haloalkyl group, a cycloalkyl group, an alkoxy group, a haloalkoxy group, and an alkyleneoxy group; the aryl moiety of the optionally substituted aryl group represented by ring A is a monocyclic or bicyclic aryl group, the heteroaryl moiety of the optionally substituted heteroaryl group represented by ring A is a 5- to 10-membered monocyclic or bicyclic heteroaryl group containing one to four heteroatom(s) independently selected from the group consisting of an oxygen atom, a sulfur atom, and a nitrogen atom;
R¹ is an optionally substituted alkyl group, an optionally substituted cycloalkyl group, an optionally substituted aliphatic heterocyclic group, an optionally substituted aryl group that may be partially hydrogenated, an optionally substituted heteroaryl group, or a carbamoyl group optionally substituted with one or two alkyl group(s), wherein substituent(s) on the optionally substituted alkyl group is/are one to three group(s) independently selected from the group consisting of a halogen atom; a hydroxy group; an oxo group; a cyano group; a cycloalkyl group; an alkoxy group; an alkanoyl group; a carbamoyl group optionally substituted with one or two alkyl group(s); a 4- to 7-membered monocyclic aliphatic heterocyclic group containing one or two heteroatom(s) independently selected from the group consisting of an oxygen atom, a sulfur atom, and a nitrogen atom; an aliphatic heterocyclic carbonyl group optionally substituted with one or two group(s) independently selected from the group consisting of a halogen atom, an alkyl group, a haloalkyl group, and an alkoxyalkyl group; an alkylsulfonyl group; an aliphatic heterocyclic sulfonyl group; and an alkyleneoxy group, substituent(s) on each of the optionally substituted cycloalkyl group, the optionally substituted aliphatic heterocyclic group, the optionally substituted aryl group that may be partially hydrogenated, and the optionally substituted heteroaryl group is/are one to three group(s) independently selected from the group consisting of a halogen atom; a hydroxy group; an oxo group; a cyano group; an alkyl group; a haloalkyl group; a cycloalkyl group; an alkoxy group; a hydroxyalkyl group; an alkoxyalkyl group; an alkanoyl group; a carbamoyl group optionally substituted with one or two alkyl group(s); a 4- to 7-membered monocyclic aliphatic heterocyclic group containing one or two heteroatom(s) independently selected from the group consisting of an oxygen atom, a sulfur atom, and a nitrogen atom; an aliphatic heterocyclic carbonyl group optionally substituted with one or two group(s) independently selected from the group consisting of a halogen atom, an alkyl group, a haloalkyl group, and an alkoxyalkyl group; an alkylsulfonyl group; an aliphatic heterocyclic sulfonyl group; and an alkyleneoxy group, the aliphatic heterocyclic moiety of each of the aliphatic heterocyclic carbonyl group and the aliphatic heterocyclic sulfonyl group with which R¹ is substituted is a 4- to 7-membered monocyclic aliphatic heterocyclic ring containing at least one nitrogen atom, and an optionally further containing one heteroatom selected from the group consisting of an oxygen atom, a sulfur atom, and a nitrogen atom, the aliphatic heterocyclic moiety of the optionally substituted aliphatic heterocyclic group represented by R² is a 4- to 7-membered monocyclic aliphatic heterocyclic ring containing one or two heteroatom(s) independently selected from the group consisting of an oxygen atom, a sulfur atom, and a nitrogen atom, and the heteroaryl moiety of the optionally substituted heteroaryl group represented by R¹ is a 5- or 6-membered monocyclic heteroaryl containing one to four heteroatom(s) independently selected from the group consisting of an oxygen atom, a sulfur atom, and a nitrogen atom;
R² is a halogen atom, an alkyl group, or an alkoxy group;
R³ is an alkyl group substituted with a substituted aryl group, wherein substituent(s) on the substituted aryl group is/are an aliphatic heterocyclic group optionally substituted with a carboxyl group (wherein the aliphatic heterocyclic group is a 4- to 8-membered monocyclic or bicyclic aliphatic heterocyclic group containing one or two heteroatom(s) independently selected from the group consisting of an oxygen atom, a sulfur atom, and a nitrogen atom) and a haloalkyl group; and
R⁴ is a hydrogen atom or an alkyl group; or
R³ and R⁴ are terminally attached to each other, and together with the nitrogen atom to which they are attached, form a group represented by above-mentioned formula [II], wherein
ring B is a 4- to 8-membered monocyclic or bicyclic aliphatic heterocyclic group that may further contain, in addition to the nitrogen atom shown in formula [II], one heteroatom selected from the group consisting of an oxygen atom, a sulfur atom, and a nitrogen atom, and may partially contain a double bond;
ring C is a monocyclic aryl group, or a 5- or 6-membered monocyclic heteroaryl group containing one to four heteroatom(s) independently selected from the group consisting of an oxygen atom, a sulfur atom, and a nitrogen atom;
R⁵ and R⁶ are each independently a group selected from the group consisting of a hydrogen atom, a halogen atom, a cyano group, an alkyl group, a haloalkyl group, a cyanoalkyl group, a hydroxyalkyl group, an alkoxyalkyl group, a carboxyl group, a carbamoyl group optionally substituted with one or two alkyl group(s), and an alkoxy group;
R⁷ is an optionally substituted alkyl group, an optionally substituted alkenyl group, an optionally substituted cycloalkyl group, an optionally substituted cycloalkenyl group, an optionally substituted aryl group, an optionally substituted heteroaryl group, an optionally substituted aliphatic heterocyclic group, an optionally substituted alkoxy group, an amino group optionally substituted with one or two alkyl group(s) optionally substituted with a carboxyl group, or a carbamoyl group optionally substituted with one or two alkyl group(s) optionally substituted with a carboxyl group, wherein substituent(s) on the optionally substituted alkyl group is/are one or two group(s) independently selected from the group consisting of a hydroxy group; an oxo group; a cyano group; an alkoxy group; an alkanoyl group; a carboxyl group; an alkoxycarbonyl group; an aliphatic heterocyclic carbonyl group optionally substituted with a carboxyl group; a heteroaryl group optionally substituted with a hydroxy or an oxo group; a 4- to 7-membered monocyclic aliphatic heterocyclic group optionally substituted with one or two oxo group(s), and containing one or two heteroatom(s) independently selected from the group consisting of an oxygen atom, a sulfur atom, and a nitrogen atom; a carbamoyl group optionally substituted with one or two group(s) independently selected from the group consisting of an alkyl group (the alkyl group is optionally substituted with a hydroxy, an alkoxy, or a carboxyl group) and a hydroxy group; an alkylsulfonyl group; an aminosulfonyl group optionally substituted with one or two alkyl group(s); an aminosulfonylaminocarbonyl group optionally substituted with one or two alkyl group(s); an alkylsulfonylaminocarbonyl group; and an amino group optionally substituted with one or two group(s) independently selected from the group consisting of an alkyl group, an alkanoyl group, and an alkylsulfonyl group, substituent(s) on each of the optionally substituted alkenyl group, the optionally substituted cycloalkyl group, the optionally substituted cycloalkenyl group, the optionally substituted aryl group, the optionally substituted heteroaryl group, the optionally substituted aliphatic heterocyclic group, and the optionally substituted alkoxy group is/are one or two group(s) independently selected from the group consisting of a hydroxy group; an oxo group; a cyano group; an alkyl group optionally substituted with a carboxyl group; an alkoxy group; an alkanoyl group; a carboxyl group; an alkoxycarbonyl group; an aliphatic heterocyclic carbonyl group optionally substituted with a carboxyl group; a heteroaryl group optionally substituted with a hydroxy or an oxo group; a 4- to 7-membered monocyclic aliphatic heterocyclic group optionally substituted with one or two oxo group(s), and containing one or two heteroatom(s) independently selected from the group consisting of an oxygen atom, a sulfur atom, and a nitrogen atom; a carbamoyl group optionally substituted with one or two group(s) independently selected from the group consisting of an alkyl group (the alkyl group is optionally substituted with a hydroxy group, an alkoxy group, or a carboxyl group) and a hydroxy group; an alkylsulfonyl group; an aminosulfonyl group optionally substituted with one or two alkyl group(s); an aminosulfonylaminocarbonyl group optionally substituted with one or two alkyl group(s); an alkylsulfonylaminocarbonyl group; and an amino group optionally substituted with one or two group(s) independently selected from the group consisting of an alkyl group, an alkanoyl group, and an alkylsulfonyl group, the aliphatic heterocyclic moiety of the aliphatic heterocyclic carbonyl group with which R⁷ is substituted is a 4- to 7-membered monocyclic aliphatic heterocyclic ring containing at least one nitrogen atom, and optionally further containing one heteroatom selected from the group consisting of an oxygen atom, a sulfur atom, and a nitrogen atom, the heteroaryl group with which R⁷ is substituted is a 5- or 6-membered monocyclic heteroaryl group containing one to four heteroatom(s) independently selected from the group consisting of an oxygen atom, a sulfur atom, and a nitrogen atom, the heteroaryl moiety of the optionally substituted heteroaryl group represented by R⁷ is a 5- or 6-membered monocyclic heteroaryl containing one to four heteroatom(s) independently selected from the group consisting of an oxygen atom, a sulfur atom, and a nitrogen atom, and the aliphatic heterocyclic moiety of the optionally substituted aliphatic heterocyclic group represented by R⁷ is a 4- to 8-membered monocyclic or bicyclic aliphatic heterocyclic ring containing one or two heteroatom(s) independently selected from the group consisting of an oxygen atom, a sulfur atom, and a nitrogen atom; and
R⁸ and R⁹ are each independently a group selected from the group consisting of a hydrogen atom, a halogen atom, a cyano group, an alkyl group, a haloalkyl group, and a haloalkoxy group.

Preferably, the invention is directed to a pyrrolidine compound or pharmaceutically acceptable salt thereof of above-mentioned formula (I), wherein:
ring A is an optionally substituted aryl group or an optionally substituted heteroaryl group, wherein substituent(s) on each of the optionally substituted aryl group and the optionally substituted heteroaryl group is/are one or two group(s) independently selected from the group consisting of a halogen atom, an alkyl group, a haloalkyl group, a cycloalkyl group, an alkoxy group, a haloalkoxy group, and an alkyleneoxy group, the aryl moiety of the optionally substituted aryl group represented by ring A is a group selected from the group consisting of a phenyl group and a naphthyl group, and the heteroaryl moiety of the optionally substituted heteroaryl group represented by ring A is a group selected from the group consisting of a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, a triazinyl group, an indolyl group, an isoindolyl group, and a benzoimidazolyl group;
R¹ is an optionally substituted alkyl group, an optionally substituted cycloalkyl group, an optionally substituted aliphatic heterocyclic group, an optionally substituted aryl group that may be partially hydrogenated, an optionally substituted heteroaryl group, or a carbamoyl group optionally substituted with one or two alkyl group(s), wherein substituent(s) on the optionally substituted alkyl group is/are one or two group(s) independently selected from the group consisting of a halogen atom; a hydroxy group; an oxo group; a cyano group; a cycloalkyl group; an alkoxy group; an alkanoyl group; a carbamoyl group optionally substituted with one or two alkyl group(s); an aliphatic heterocyclic group; an aliphatic heterocyclic carbonyl group optionally substituted with one or two group(s) independently selected from the group consisting of a halogen atom, an alkyl group, a haloalkyl group, and an alkoxyalkyl group; an alkylsulfonyl group; an aliphatic heterocyclic sulfonyl group; and an alkyleneoxy group, substituent(s) on each of the optionally substituted cycloalkyl group, the optionally substituted aliphatic heterocyclic group, the optionally substituted aryl group that may be partially hydrogenated, and the optionally substituted heteroaryl group is/are one or two group(s) independently selected from the group consisting of a halogen atom; a hydroxy group; an oxo group; a cyano group; an alkyl group; a haloalkyl group; a cycloalkyl group; an alkoxy group; a hydroxyalkyl group; an alkoxyalkyl group; an alkanoyl group; a carbamoyl group optionally substituted with one or two alkyl group(s); an aliphatic heterocyclic group; an aliphatic heterocyclic carbonyl group optionally substituted with one or two group(s) independently selected from the group consisting of a halogen atom, an alkyl group, a haloalkyl group, and an alkoxyalkyl group; an alkylsulfonyl group; an aliphatic heterocyclic sulfonyl group; and an alkyleneoxy group, the aliphatic heterocyclic group with which R¹ is substituted is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, and an homomorpholinyl group, the aliphatic heterocyclic moiety of each of the aliphatic heterocyclic carbonyl group and the aliphatic heterocyclic sulfonyl group with which R¹ is substituted is a group selected from the group consisting of an azetidinyl group, a pyrrolidinyl group, an imidazolinyl group, a thiazolidinyl group, an isothiazolidinyl group, a piperidinyl group, a piperazinyl group, a morpholinyl group, a thiomorpholinyl group, a homopiperazinyl group, and a homomorpholinyl group, the aryl moiety of the optionally substituted aryl group that may be partially hydrogenated represented by Ill is a group selected from the group consisting of a phenyl group, a naphthyl group, a dihydrophenyl group, an indanyl group, and a tetrahydronaphthyl group, the aliphatic heterocyclic moiety of the optionally substituted aliphatic heterocyclic group represented by R¹ is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, and a homomorpholinyl group, and the heteroaryl moiety of the optionally substituted heteroaryl group represented by R¹ is a group selected from the group consisting of a pyrrolyl group, a furanyl group, a thienyl group, an imidazolyl group, a pyrazolyl group, an oxazolyl group, an isoxazolyl group, a thiazolyl, tetrazolyl, oxadiazolyl, pyridinyl, pyrazinyl group, a pyrimidinyl group, a pyridazinyl group, a thiazinyl group, and a triazinyl group;
R² is a halogen atom, an alkyl group, or an alkoxy group;
R³ is an alkyl group substituted with a substituted aryl group, wherein substituent(s) on the substituted aryl group is/are an aliphatic heterocyclic group optionally substituted with a carboxyl group (wherein the aliphatic heterocyclic group is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, a homomorpholinyl group, a 3-azabicyclo[3.1.0]hexyl group, and an octahydropyrrolo[3,4-c]pyrrolyl group) and a haloalkyl group; and
R⁴ is a hydrogen atom or an alkyl group; or
R³ and R⁴ are terminally attached to each other, and together with the nitrogen atom to which they are attached, form a group represented by above-mentioned formula [II], wherein
ring B is a group selected from the group consisting of an azetidinyl group, a pyrrolidinyl group, an imidazolinyl group, a thiazolidinyl group, an isothiazolidinyl group, a piperidinyl group, a piperazinyl group, a morpholinyl group, a thiomorpholinyl group, a tetrahydropyridinyl group, a homopiperazinyl group, a homomorpholinyl group, a 3-azabicyclo[3.1.0]hexyl group, and an octahydropyrrolo[3,4-c]pyrrolyl group;
ring C is a group selected from the group consisting of a phenyl group, a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, and a triazinyl group;
R⁵ and R⁶ are each independently a group selected from the group consisting of a hydrogen atom, a halogen atom, a cyano group, an alkyl group, a haloalkyl group, a cyanoalkyl group, a hydroxyalkyl group, an alkoxyalkyl group, a carboxyl group, a carbamoyl group optionally substituted with one or two alkyl group(s), and an alkoxy group;
R⁷ is an optionally substituted alkyl group, an optionally substituted alkenyl group, an optionally substituted cycloalkyl group, an optionally substituted cycloalkenyl group, an optionally substituted aryl group, an optionally substituted heteroaryl group, an optionally substituted aliphatic heterocyclic group, an optionally substituted alkoxy group, an amino group optionally substituted with one or two alkyl group(s) optionally substituted with a carboxyl group, or a carbamoyl group optionally substituted with one or two alkyl group(s) optionally substituted with a carboxyl group, wherein substituent(s) on the optionally substituted alkyl group is/are one or two group(s) independently selected from the group consisting of a hydroxy group; an oxo group; a cyano group; an alkoxy group; an alkanoyl group; a carboxyl group; an alkoxycarbonyl group; an aliphatic heterocyclic carbonyl group optionally substituted with a carboxyl group; a heteroaryl group optionally substituted with a hydroxy group or an oxo group; an aliphatic heterocyclic group optionally substituted with one or two oxo group(s); a carbamoyl group optionally substituted with one or two group(s) independently selected from the group consisting of an alkyl group (the alkyl group is optionally substituted with a hydroxy group, an alkoxy group, or a carboxyl group) and a hydroxy group; an alkylsulfonyl group; an aminosulfonyl group optionally substituted with one or two alkyl group(s); an aminosulfonylaminocarbonyl group optionally substituted with one or two alkyl group(s); an alkylsulfonylaminocarbonyl group; and an amino group optionally substituted with one or two group(s) independently selected from the group consisting of an alkyl group, an alkanoyl group, and an alkylsulfonyl group, substituent(s) on each of the optionally substituted alkenyl group, the optionally substituted cycloalkyl group, the optionally substituted cycloalkenyl group, the optionally substituted aryl group, the optionally substituted heteroaryl group, the optionally substituted aliphatic heterocyclic group, and the optionally substituted alkoxy group is/are one or two group(s) independently selected from the group consisting of a hydroxy group; an oxo group; a cyano group; an alkyl group optionally substituted with a carboxyl group; an alkoxy group; an alkanoyl group; a carboxyl group; an alkoxycarbonyl group; an aliphatic heterocyclic carbonyl group optionally substituted with a carboxyl group; a heteroaryl group optionally substituted with a hydroxy group or an oxo group; an aliphatic heterocyclic group optionally substituted with one or two oxo group(s); a carbamoyl group optionally substituted with one or two group(s) independently selected from the group consisting of an alkyl group (the alkyl group is optionally substituted with a hydroxy group, an alkoxy group, or a carboxyl group) and a hydroxy group; an alkylsulfonyl group; an aminosulfonyl group optionally substituted with one or two alkyl group(s); an aminosulfonylaminocarbonyl group optionally substituted with one or two alkyl group(s); an alkylsulfonylaminocarbonyl group; and an amino group optionally substituted with one or two group(s) independently selected from the group consisting of an alkyl group, an alkanoyl group, and an alkylsulfonyl group, the aliphatic heterocyclic moiety of the aliphatic heterocyclic carbonyl group with which R⁷ is substituted is a group selected from the group consisting of an azetidinyl group, a pyrrolidinyl group, an imidazolinyl group, a thiazolidinyl group, an isothiazolidinyl group, a piperidinyl group, a piperazinyl group, a morpholinyl group, a thiomorpholinyl group, a homopiperazinyl group, and a homomorpholinyl group, the heteroaryl group with which R⁷ is substituted is a group selected from the group consisting of a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, and a triazinyl group, the aliphatic heterocyclic group with which R⁷ is substituted is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, and a homomorpholinyl group, the heteroaryl moiety of the optionally substituted heteroaryl group represented by R⁷ is a group selected from the group consisting of a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, and a triazinyl group, and the aliphatic heterocyclic moiety of the optionally substituted aliphatic heterocyclic group represented by R⁷ is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, a homomorpholinyl group, a 3-azabicyclo[3.1.0]hexyl group, and an octahydropyrrolo[3,4-c]pyrrolyl group; and
R⁸ and R⁹ are each independently a group selected from the group consisting of a hydrogen atom, a halogen atom, a cyano group, an alkyl group, a haloalkyl group, and a haloalkoxy group.

Preferably, the invention is directed to a pyrrolidine compound or pharmaceutically acceptable salt thereof of above-mentioned formula (I), wherein:
ring A is a phenyl group or a naphthyl group each optionally substituted with one or two group(s) independently selected from the group consisting of a halogen atom, an alkyl group, a haloalkyl group, a cycloalkyl group, an alkoxy group, a haloalkoxy group, and an alkyleneoxy group; or a heteroaryl group optionally substituted with one or two group(s) independently selected from the group consisting of a halogen atom and an alkoxy group, wherein the heteroaryl group is a group selected from the group consisting of a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, a triazinyl group, an indolyl group, an isoindolyl group, and a benzoimidazolyl group;
R¹ is
(1) an alkyl group optionally substituted with one or two group(s) independently selected from the group consisting of a halogen atom; a hydroxy group; a cycloalkyl group; an alkoxy group; an aliphatic heterocyclic group; an aliphatic heterocyclic carbonyl group optionally substituted with one or two group(s) independently selected from the group consisting of a halogen atom, an alkyl group, a haloalkyl group, and an alkoxyalkyl group; an aliphatic heterocyclic sulfonyl group; and a carbamoyl group optionally substituted with one or two alkyl group(s) (wherein the aliphatic heterocyclic group is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, and a homomorpholinyl group, and the aliphatic heterocyclic moiety of each of the aliphatic heterocyclic carbonyl group and the aliphatic heterocyclic sulfonyl group is a group selected from the group consisting of an azetidinyl group, a pyrrolidinyl group, an imidazolinyl group, a thiazolidinyl group, an isothiazolidinyl group, a piperidinyl group, a piperazinyl group, a morpholinyl group, a thiomorpholinyl group, a homopiperazinyl group, and a homomorpholinyl group),
(2) a monocyclic cycloalkyl group optionally substituted with one or two group(s) independently selected from the group consisting of a halogen atom, a hydroxy group, an oxo group, a cyano group, an alkyl group, an alkoxy group, an alkoxyalkyl group, and an alkyleneoxy group,
(3) an adamantyl group optionally substituted with a hydroxy group,
(4) an aliphatic heterocyclic group optionally substituted with a group selected from the group consisting of an alkyl group, a hydroxyalkyl group, a haloalkyl group, an alkanoyl group, and an alkylsulfonyl group (wherein the aliphatic heterocyclic group is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, and a homomorpholinyl group),
(5) a group selected from the group consisting of a phenyl group, a naphthyl group, a dihydrophenyl group, an indanyl group, and a tetrahydronaphthyl group,
(6) a heteroaryl group which is optionally substituted with a group selected from the group consisting of a cyano group, an alkyl group, an alkoxy group, and a carbamoyl group (wherein the heteroaryl group is a group selected from the group consisting of a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, and a triazinyl group),
(7) a carbamoyl group, or
(8) a mono-alkylcarbamoyl group;
R² is a halogen atom, an alkyl group, or an alkoxy group;
R³ is an alkyl group substituted with a substituted phenyl group, wherein substituent(s) on the substituted phenyl group is/are an aliphatic heterocyclic group substituted with a carboxyl group (wherein the aliphatic heterocyclic group is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, a homomorpholinyl group, a 3-azabicyclo[3.1.0]hexyl group, and an octahydropyrrolo[3,4-c]pyrrolyl group) and a haloalkyl group; and
R⁴ is an alkyl group; or
R³ and R⁴ are terminally attached to each other, and together with the nitrogen atom to which they are attached, form a group represented by above-mentioned formula [II], wherein ring B is a group selected from the group consisting of an azetidinyl group, an imidazolinyl group, a thiazolidinyl group, an isothiazolidinyl group, a piperazinyl group, a morpholinyl group, a thiomorpholinyl group, a tetrahydropyridinyl group, a homopiperazinyl group, a homomorpholinyl group, a 3-azabicyclo[3.1.0]hexyl group, and an octahydropyrrolo[3,4-c]pyrrolyl group, and both R⁵ and R⁶ represent hydrogen atoms, or ring B is a piperidinyl group, and R⁵ and R⁶ are each a group independently selected from the group consisting of a hydrogen atom, a cyano group, and an alkoxyalkyl group, or ring B is a pyrrolidinyl group, and R⁵ and R⁶ are each a group independently selected from the group consisting of a hydrogen atom, a halogen atom, a cyano group, an alkyl group, a haloalkyl group, a cyanoalkyl group, a hydroxyalkyl group, an alkoxyalkyl group, a carboxyl group, a carbamoyl group optionally substituted with one or two alkyl group(s), and an alkoxy group;
ring C is a group selected from the group consisting of a phenyl group, a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, and a triazinyl group;
R⁷ is:
(1) an alkyl group optionally substituted with a carboxyl group,
(2) an alkenyl group optionally substituted with a carboxyl group,
(3) a 3- to 7-membered monocyclic cycloalkyl group optionally substituted with a carboxyl group,
(4) a 3- to 7-membered monocyclic cycloalkenyl group optionally substituted with a carboxyl group,
(5) a phenyl group optionally substituted with a carboxyl group,
(6) a heteroaryl group which is optionally substituted with a carboxyl group or an alkyl group optionally substituted with a carboxyl group (wherein the heteroaryl group is a group selected from the group consisting of a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, and a triazinyl group),
(7) an aliphatic heterocyclic group optionally substituted with one or two group(s) independently selected from the group consisting of a hydroxy group; an oxo group; a cyano group; an alkyl group optionally substituted with a carboxyl group; an alkoxy group; an alkanoyl group; a carboxyl group; an alkoxycarbonyl group; a carbamoyl group optionally substituted with one or two group(s) independently selected from the group consisting of an alkyl group (the alkyl group is optionally substituted with a hydroxy group, an alkoxy group, or a carboxyl group) and a hydroxy group; an alkylsulfonylaminocarbonyl group; an aliphatic heterocyclic carbonyl group optionally substituted with a carboxyl group (wherein the aliphatic heterocyclic ring is a group selected from the group consisting of an azetidinyl group, a pyrrolidinyl group, an imidazolinyl group, a thiazolidinyl group, an isothiazolidinyl group, a piperidinyl group, a piperazinyl group, a morpholinyl group, a thiomorpholinyl group, a homopiperazinyl group, and a homomorpholinyl group); an amino group optionally substituted with one or two group(s) independently selected from the group consisting of an alkyl group, an alkanoyl group, and an alkylsulfonyl group; an aliphatic heterocyclic group optionally substituted with one or two oxo group(s) (wherein the aliphatic heterocyclic group is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, and a homomorpholinyl group); an alkylsulfonyl group; a heteroaryl group (wherein the heteroaryl group is a group selected from the group consisting of a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, and a triazinyl group); and an aminosulfonyl group optionally substituted with one or two alkyl group(s) (wherein the aliphatic heterocyclic group is a group selected from the group consisting of an azetidinyl group, a pyrrolidinyl group, a tetrahydrofuranyl group, an imidazolinyl group, a thiazolidinyl group, an isothiazolidinyl group, a piperidinyl group, a piperazinyl group, a morpholinyl group, a thiomorpholinyl group, a tetrahydropyranyl group, a homopiperazinyl group, a homomorpholinyl group, a 3-azabicyclo[3.1.0]hexyl group, and an octahydropyrrolo[3,4-c]pyrrolyl group),
(8) an alkoxy group optionally substituted with a group selected from the group consisting of a cyano group; a carboxyl group; a heteroaryl group optionally substituted with a hydroxy group or an oxo group (wherein the heteroaryl group is a group selected from the group consisting of a pyrrolyl group, a furanyl group, a thienyl group, an imidazolyl group, a pyrazolyl group, an oxazolyl group, an isoxazolyl group, a thiazolyl group, a tetrazolyl group, an oxadiazolyl group, a pyridinyl group, a pyrazinyl group, a pyrimidinyl group, a pyridazinyl group, a thiazinyl group, and a triazinyl group); an aminosulfonylaminocarbonyl group optionally substituted with one or two alkyl group(s); and an alkylsulfonylaminocarbonyl group,
(9) an amino group which is optionally substituted with one or two alkyl group(s) optionally substituted with a carboxyl group, or
(10) a carbamoyl group; and
R⁸ and R⁹ are each independently a group selected from the group consisting of a hydrogen atom, a halogen atom, a cyano group, an alkyl group, a haloalkyl group, and a haloalkoxy group.

Preferably, the invention is directed to a pyrrolidine compound or pharmaceutically acceptable salt thereof of above-mentioned formula (I), wherein
ring A is a phenyl group optionally substituted with one or two groups independently selected from the group consisting of a halogen atom, an alkyl group, a haloalkyl group, a cycloalkyl group, an alkoxy group, a haloalkoxy group, and an alkyleneoxy group, or a pyridinyl group optionally substituted with a group selected from the group consisting of a halogen atom and an alkoxy group;
R¹ is
(1) an alkyl group optionally substituted with one or two group(s) independently selected from the group consisting of a halogen atom; a hydroxy group; a 3- to 7-membered monocyclic cycloalkyl group; an alkoxy group; a tetrahydropyranyl group; an aliphatic heterocyclic carbonyl group optionally substituted with one or two group(s) independently selected from the group consisting of a halogen atom, an alkyl group, a haloalkyl group, and an alkoxyalkyl group (wherein the aliphatic heterocyclic ring is a group selected from the group consisting of a pyrrolidinyl group, a piperidinyl group, and a morpholinyl group); a pyrrolidinylsulfonyl group; and a carbamoyl group optionally substituted with one or two alkyl group(s),
(2) a 3- to 7-membered monocyclic cycloalkyl group optionally substituted with one or two group(s) independently selected from the group consisting of a halogen atom, a hydroxy group, an oxo group, a cyano group, an alkyl group, an alkoxy group, an alkoxyalkyl group, and an alkyleneoxy group,
(3) an adamantyl group optionally substituted with a hydroxy group,
(4) an aliphatic heterocyclic group optionally substituted with a group selected from the group consisting of an alkyl group, a hydroxyalkyl group, a haloalkyl group, an alkanoyl group, and an alkylsulfonyl group (wherein the aliphatic heterocyclic group is a group selected from the group consisting of a tetrahydrofuranyl group, a tetrahydropyranyl group, and a piperidinyl group),
(5) an indanyl group,
(6) a heteroaryl group which is optionally substituted with a group selected from the group consisting of a cyano group, an alkyl group, an alkoxy group, and a carbamoyl group (wherein the heteroaryl group is a group selected from the group consisting of a pyridazinyl group, a pyridinyl group, and a pyrimidinyl group),
(7) a carbamoyl group, or
(8) a mono-alkylcarbamoyl group;
R² is a halogen atom, a C₁₋₃ alkyl group, or an alkoxy group;
R³ is an alkyl group substituted with a substituted phenyl group, wherein substituent(s) on the substituted phenyl group is/are a piperidinyl group substituted with a carboxyl group, and a haloalkyl group; and
R⁴ is an alkyl group; or
R³ and R⁴ are terminally attached to each other, and together with the nitrogen atom to which they are attached, form a group represented by above-mentioned formula [II], wherein
ring B is a group selected from the group consisting of an azetidinyl group, a tetrahydropyridinyl group, a piperazinyl group, a homopiperazinyl group, and an octahydropyrrolo[3,4-c]pyrrolyl group, and both R⁵ and R⁶ represent hydrogen atoms, or ring B is a piperidinyl group, and R⁵ and R⁶ are each a group independently selected from the group consisting of a hydrogen atom, a cyano group, and an alkoxyalkyl group, or ring B is a pyrrolidinyl group, and R⁵ and R⁶ are each a group independently selected from the group consisting of a hydrogen atom, a halogen atom, a cyano group, an alkyl group, a haloalkyl group, a cyanoalkyl group, a hydroxyalkyl group, an alkoxyalkyl group, a carboxyl group, carbamoyl group optionally substituted with one or two alkyl group(s), and an alkoxy group;
ring C is a phenyl group or a pyridinyl group;
R⁷ is:
(1) an alkyl group optionally substituted with a carboxyl group,
(2) an alkenyl group optionally substituted with a carboxyl group,
(3) a 3- to 7-membered monocyclic cycloalkyl group optionally substituted with a carboxyl group,
(4) a 3- to 7-membered monocyclic cycloalkenyl group optionally substituted with a carboxyl group,
(5) a phenyl group optionally substituted with a carboxyl group,
(6) a heteroaryl group which is optionally substituted with a carboxyl group or an alkyl group optionally substituted with a carboxyl group (wherein the heteroaryl group is a group selected from the group consisting of an oxazolyl group and a pyrazolyl group),
(7) an aliphatic heterocyclic group optionally substituted with one or two group(s) independently selected from the group consisting of a hydroxy group; an oxo group; a cyano group; an alkyl group optionally substituted with a carboxyl group; an alkoxy group; an alkanoyl group; a carboxyl group; an alkoxycarbonyl group; a carbamoyl group optionally substituted with one or two group(s) independently selected from the group consisting of an alkyl group (the alkyl group is optionally substituted with a hydroxy group, an alkoxy group, or a carboxyl group) and a hydroxy group; an alkylsulfonylaminocarbonyl group; a pyrrolidinylcarbonyl group optionally substituted with a carboxyl group; an amino group optionally substituted with one or two group(s) independently selected from the group consisting of an alkyl group, an alkanoyl group, and an alkylsulfonyl group; an aliphatic heterocyclic group optionally substituted with one or two oxo group(s) (wherein the aliphatic heterocyclic group is a group selected from the group consisting of a pyrrolidinyl group and an isothiazolidinyl group); an alkylsulfonyl group; a tetrazolyl group; and an aminosulfonyl group optionally substituted with one or two alkyl group(s) (wherein the aliphatic heterocyclic group is a group selected from the group consisting of an azetidinyl group, a pyrrolidinyl group, a piperidinyl group, a morpholinyl group, a thiomorpholinyl group, a piperazinyl group, and a 3-azabicyclo[3.1.0]hexyl group),
(8) an alkoxy group optionally substituted with a group selected from the group consisting of a cyano group; a carboxyl group; a heteroaryl group optionally substituted with a hydroxy group or an oxo group (wherein the heteroaryl group is a group selected from the group consisting of an isoxazolyl group, an oxadiazolyl group, and a tetrazolyl group); an aminosulfonylaminocarbonyl group optionally substituted with one or two alkyl group(s); and an alkylsulfonylaminocarbonyl group,
(9) an amino group which is optionally substituted with one or two alkyl group(s) optionally substituted with a carboxyl group, or
(10) a carbamoyl group; and
R⁸ and R⁹ are each independently a group selected from the group consisting of a hydrogen atom, a halogen atom, a cyano group, an alkyl group, a haloalkyl group, and a haloalkoxy group.

Preferably, the invention is directed to a pyrrolidine compound or pharmaceutically acceptable salt thereof of above-mentioned formula (I), wherein R³ and R⁴ are terminally attached to each other, and together with the nitrogen atom to which they are attached, form a group represented by above-mentioned formula [II].

Preferably, the invention is directed to a pyrrolidine compound or pharmaceutically acceptable salt thereof of above-mentioned formula (I), wherein
R¹ is
(1) a 3- to 7-membered monocyclic cycloalkyl group optionally substituted with one or two group(s) independently selected from the group consisting of a halogen atom, a hydroxy group, an oxo group, a cyano group, an alkyl group, an alkoxy group, an alkoxyalkyl group, and an alkyleneoxy group;
(2) an adamantyl group optionally substituted with a hydroxy group; or
(3) an aliphatic heterocyclic group optionally substituted with a group selected from the group consisting of an alkyl group, a hydroxyalkyl group, a haloalkyl group, an alkanoyl group, and an alkylsulfonyl group (wherein the aliphatic heterocyclic group is a group selected from the group consisting of a tetrahydrofuranyl group, a tetrahydropyranyl group, and a piperidinyl group).

Preferably, the invention is directed to a pyrrolidine compound or pharmaceutically acceptable salt thereof of formula (I), wherein R³ and R⁴ are terminally attached to each other, and together with the nitrogen atom to which they are attached, form a group represented by above-mentioned formula [II] wherein ring B is a pyrrolidinyl group.

Preferably, the invention is directed to a pyrrolidine compound or pharmaceutically acceptable salt thereof of above-mentioned formula (I), wherein R¹ is a 3- to 7-membered monocyclic cycloalkyl group optionally substituted with one or two group(s) independently selected from the group consisting of a halogen atom, a hydroxy group, an oxo group, a cyano group, an alkyl group, an alkoxy group, an alkoxyalkyl group, and an alkyleneoxy group.

Preferably, the invention is directed to a pyrrolidine compound or pharmaceutically acceptable salt thereof of above-mentioned formula (I), wherein ring A is a phenyl group optionally substituted with one or two group(s) independently selected from the group consisting of a halogen atom, an alkyl group, a haloalkyl group, a cycloalkyl group, an alkoxy group, a haloalkoxy group, and an alkyleneoxy group;
R¹ is a 3- to 7-membered monocyclic cycloalkyl group optionally substituted with one or two group(s) independently selected from the group consisting of a halogen atom, a hydroxy group, an oxo group, a cyano group, an alkyl group, an alkoxy group, an alkoxyalkyl group, and an alkyleneoxy group;
R² is a halogen atom or an alkoxy group;
R³ and R⁴ are terminally attached to each other, and together with the nitrogen atom to which they are attached, form a group represented by above-mentioned formula [II], wherein ring B is a pyrrolidinyl group, and R⁵ and R⁶ are each a group independently selected from the group consisting of a hydrogen atom, a halogen atom, a cyano group, an alkyl group, a haloalkyl group, a cyanoalkyl group, a hydroxyalkyl group, an alkoxyalkyl group, a carboxyl group, a carbamoyl group optionally substituted with one or two alkyl group(s), and an alkoxy group; ring C is a phenyl group;
R⁷ is
(1) an alkyl group optionally substituted with a carboxyl group,
(2) an alkenyl group optionally substituted with a carboxyl group,
(3) a 3- to 7-membered monocyclic cycloalkyl group optionally substituted with a carboxyl group,
(4) a 3- to 7-membered monocyclic cycloalkenyl group optionally substituted with a carboxyl group,
(5) a phenyl group optionally substituted with a carboxyl group,
(6) a heteroaryl group which is optionally substituted with a carboxyl group or an alkyl group optionally substituted with a carboxyl group (wherein the heteroaryl group is a group selected from the group consisting of an oxazolyl group and a pyrazolyl group),
(7) an aliphatic heterocyclic group optionally substituted with one or two group(s) independently selected from the group consisting of a hydroxy group; an oxo group; a cyano group; an alkyl group optionally substituted with a carboxyl group; an alkoxy group; an alkanoyl group; a carboxyl group; an alkoxycarbonyl group; a carbamoyl group optionally substituted with one or two group(s) independently selected from the group consisting of an alkyl group (the alkyl group is optionally substituted with a hydroxy group, an alkoxy group, or a carboxyl group) and a hydroxy group; an alkylsulfonylaminocarbonyl group; a pyrrolidinylcarbonyl group optionally substituted with a carboxyl group; an amino group optionally substituted with one or two group(s) independently selected from the group consisting of an alkyl group, an alkanoyl group, and an alkylsulfonyl group; an aliphatic heterocyclic group optionally substituted with one or two oxo group(s) (wherein the aliphatic heterocyclic group is a group selected from the group consisting of a pyrrolidinyl group and an isothiazolidinyl group); an alkylsulfonyl group; a tetrazolyl group; and an aminosulfonyl group optionally substituted with one or two alkyl group(s) (wherein the aliphatic heterocyclic group is a group selected from the group consisting of an azetidinyl group, a pyrrolidinyl group, a piperidinyl group, a morpholinyl group, a thiomorpholinyl group, a piperazinyl group, and a 3-azabicyclo[3.1.0]hexyl group),
(8) an alkoxy group optionally substituted with a group selected from the group consisting of a cyano group; a carboxyl group; a heteroaryl group optionally substituted with a hydroxy group or an oxo group (wherein the heteroaryl group is a group selected from the group consisting of an isoxazolyl group, an oxadiazolyl group, and a tetrazolyl group); an aminosulfonylaminocarbonyl group optionally substituted with one or two alkyl group(s); and an alkylsulfonylaminocarbonyl group,
(9) an amino group which is optionally substituted with one or two alkyl group(s) optionally substituted with a carboxyl group, or
(10) a carbamoyl group; and
R⁸ and R⁹ are each independently a group selected from the group consisting of a hydrogen atom, a halogen atom, a cyano group, an alkyl group, a haloalkyl group, and a haloalkoxy group.

Preferably, the invention is directed to a pyrrolidine compound or pharmaceutically acceptable salt thereof of above-mentioned formula (I), wherein:
ring A is a phenyl group optionally substituted with one or two group(s) independently selected from the group consisting of a halogen atom, an alkyl group, a haloalkyl group, a cycloalkyl group, an alkoxy group, a haloalkoxy group, and an alkyleneoxy group;
R¹ is a 3- to 7-membered monocyclic cycloalkyl group optionally substituted with one or two group(s) independently selected from the group consisting of a halogen atom, a hydroxy group, an oxo group, a cyano group, an alkyl group, an alkoxy group, an alkoxyalkyl group, and an alkyleneoxy group;
R² is a halogen atom or an alkoxy group;
R³ and R⁴ are terminally attached to each other, and together with the nitrogen atom to which they are attached, form a group represented by above-mentioned formula [II], wherein
ring B is a pyrrolidinyl group, and R⁵ and R⁶ are each a group independently selected from the group consisting of a hydrogen atom, a halogen atom, a cyano group, an alkyl group, a haloalkyl group, a cyanoalkyl group, a hydroxyalkyl group, an alkoxyalkyl group, a carboxyl group, a carbamoyl group optionally substituted with one or two alkyl group(s), and an alkoxy group;
ring C is a phenyl group;
R⁷ is an aliphatic heterocyclic group optionally substituted with one or two group(s) independently selected from the group consisting of a hydroxy group; an oxo group; a cyano group; an alkyl group optionally substituted with a carboxyl group; an alkoxy group; an alkanoyl group; a carboxyl group; an alkoxycarbonyl group; a carbamoyl group optionally substituted with one or two group(s) independently selected from the group consisting of an alkyl group (the alkyl group is optionally substituted with a hydroxy group, an alkoxy group, or a carboxyl group) and a hydroxy group; an alkylsulfonylaminocarbonyl group; a pyrrolidinylcarbonyl group optionally substituted with a carboxyl group; an amino group optionally substituted with one or two group(s) independently selected from the group consisting of an alkyl group, an alkanoyl group, and an alkylsulfonyl group; an aliphatic heterocyclic group optionally substituted with one or two oxo group(s) (wherein the aliphatic heterocyclic group is a group selected from the group consisting of a pyrrolidinyl group and an isothiazolidinyl group); an alkylsulfonyl group; a tetrazolyl group; and an aminosulfonyl group optionally substituted with one or two alkyl group(s) (wherein the aliphatic heterocyclic group is a group selected from the group consisting of an azetidinyl group, a pyrrolidinyl group, a piperidinyl group, a morpholinyl group, a thiomorpholinyl group, a piperazinyl group, and a 3-azabicyclo[3.1.0]hexyl group); and
R⁸ and R⁹ are each independently a group selected from the group consisting of a hydrogen atom, a halogen atom, a cyano group, an alkyl group, a haloalkyl group, and a haloalkoxy group.

Preferably, the invention is directed to a pyrrolidine compound or pharmaceutically acceptable salt thereof of above-mentioned formula (I), wherein
ring A is a phenyl group optionally substituted with an alkoxy group;
R¹ is an alkyl group; an aliphatic heterocyclic group (wherein the aliphatic heterocyclic group is a group selected from the group consisting of a tetrahydrofuranyl group, a tetrahydropyranyl group, and a piperidinyl group); a 3- to 7-membered monocyclic cycloalkyl group optionally substituted with a group selected from the group consisting of an alkoxy group and a cyano group; or a heteroaryl group optionally substituted with an alkyl group (wherein the heteroaryl group is a group selected from the group consisting of a pyridazinyl group, a pyridinyl group, and a pyrimidinyl group);
R² is a halogen atom or an alkoxy group;
R³ and R⁴ are terminally attached to each other, and together with the nitrogen atom to which they are attached, form a group represented by above-mentioned formula [II] wherein
ring B is selected from the group consisting of an azetidinyl group, a pyrrolidinyl group, a piperidinyl group, a tetrahydropyridinyl group, and a piperazinyl group, and R⁵ and R⁶ are hydrogen atoms, or
ring B is a pyrrolidinyl group, R⁵ is an alkoxyalkyl group, and R⁶ is a hydrogen atom or a halogen atom;
ring C is a phenyl group;
R⁷ is an aliphatic heterocyclic group substituted with a carboxyl group (wherein the aliphatic heterocyclic group is a group selected from the group consisting of an azetidinyl group, a pyrrolidinyl group, a piperidinyl group, a morpholinyl group, a thiomorpholinyl group, a piperazinyl group, and a 3-azabicyclo[3.1.0]hexyl group);
R⁸ is a halogen atom or a haloalkyl group; and
R⁹ is a hydrogen atom.

Preferably, the invention is directed to a pyrrolidine compound or pharmaceutically acceptable salt thereof of above-mentioned formula (I), wherein
ring A is a phenyl group optionally substituted with an alkoxy group;
R¹ is a tetrahydropyranyl group; or a 5- or 6-membered monocyclic cycloalkyl group optionally substituted with a group selected from the group consisting of an alkoxy group and a cyano group;
R² is a halogen atom or an alkoxy group;
R³ and R⁴ are terminally attached to each other, and together with the nitrogen atom to which they are attached, form a group represented by above-mentioned formula [II] wherein
ring B is a pyrrolidinyl group, R⁵ is an alkoxyalkyl group, and R⁶ is a hydrogen atom or a halogen atom;
ring C is a phenyl group;
R⁷ is a piperidinyl group substituted with a carboxyl group;
R⁸ is a halogen atom or a haloalkyl group; and
R⁹ is a hydrogen atom.

Preferably, the invention is directed to a pyrrolidine compound or pharmaceutically acceptable salt thereof of above-mentioned formula (I), wherein
ring A is a phenyl group optionally substituted with an alkoxy group;
R¹ is a 5- or 6-membered monocyclic cycloalkyl group optionally substituted with a group selected from the group consisting of an alkoxy group and a cyano group;
R² is a halogen atom or an alkoxy group;
R³ and R⁴ are terminally attached to each other, and together with the nitrogen atom to which they are attached, form a group represented by above-mentioned formula [II] wherein
ring B is a pyrrolidinyl group, R⁵ is an alkoxyalkyl group, and R⁶ is a hydrogen atom or a halogen atom;
ring C is a phenyl group;
R⁷ is a piperidinyl group substituted with a carboxyl group;
R⁸ is a halogen atom or a haloalkyl group; and
R⁹ is a hydrogen atom.

Preferably, the pyrrolidine compound or pharmaceutically acceptable salt thereof of the invention is selected from the group consisting of:
1-{2-[(3S,5S)-1-{[(3R,4R)-1-(trans-4-ethoxycyclohexyl)-3-methoxy-4-(4-methoxyphenyl)pyrrolidin-3-yl]carbonyl}-5- (ethoxymethyl)pyrrolidin-3-yl]-5-(trifluoromethyl)phenyl}piperidine-4-carboxylic acid;
1-{2-[(3S,5S)-1-{[(3R,4R)-3-fluoro-4-(4-methoxyphenyl)-1-(2-methylpyridin-4-yl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,5S)-1-{[(3R,4R)-3-fluoro-4-(4-methoxyphenyl)-1-(2-methylpyrimidin-4-yl)pyrrolidin-3-yl]carbonyl}-5- (methoxymethyl)pyrrolidin-3-yl]-5-(trifluoromethyl)phenyl}piperidine-4-carboxylic acid;
1-{5-fluoro-2-[(3S,5S)-1-{[(3R,4R)-3-fluoro-1-(trans-4-methoxycyclohexyl)-4-(4-methoxyphenyl)pyrrolidin-3-yl]carbonyl}-5-(methoxymethyl)pyrrolidin-3-yl]phenyl}piperidine-4-carboxylic acid;
1-{2-[(3S,5S)-1-{[(3R,4R)-1-(trans-4-ethoxycyclohexyl)-3-methoxy-4-(4-methoxyphenyl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,5S)-1-{[(3R,4R)-1-(trans-4-cyanocyclohexyl)-3-fluoro-4-(4-methoxyphenyl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,4S)-1-{[(3R,4R)-1-(trans-4-ethoxycyclohexyl)-3-methoxy-4-(4-methoxyphenyl)pyrrolidin-3-yl]carbonyl}-4-fluoro-4-(methoxymethyl)pyrrolidin-3-yl]-5-(trifluoromethyl)phenyl}piperidine-4-carboxylic acid;
1-{2-[(3S,4R)-1-{[(3R,4R)-1-(trans-4-ethoxycyclohexyl)-3-methoxy-4-(4-methoxyphenyl)pyrrolidin-3-yl]carbonyl}-4- (methoxymethyl)pyrrolidin-3-yl]-5-(trifluoromethyl)phenyl}piperidine-4-carboxylic acid;
1-{2-[(3S,5S)-1-{[(3R,4R)-1-cyclohexyl-3-fluoro-4-(4-methoxyphenyl)pyrrolidin-3-yl]carbonyl}-5-(methoxymethyl)pyrrolidin-3- yl]-5-(trifluoromethyl)phenyl}piperidine-4-carboxylic acid;
1-{2-[(3S,5S)-1-{[(3R,4R)-3-fluoro-4-(4-methoxyphenyl)-1-(tetrahydro-2H-pyran-4-yl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,5S)-1-{[(3R,4R)-3-fluoro-1-(trans-4-methoxycyclohexyl)-4-(4-methoxyphenyl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,5S)-1-{[(3R,4R)-1-cyclopentyl-3-fluoro-4-(4-methoxyphenyl)pyrrolidin-3-yl]carbonyl}-5-(methoxymethyl)pyrrolidin-3- yl]-5-(trifluoromethyl)phenyl}piperidine-4-carboxylic acid;
1-{2-[(3S,4R)-1-{[(3R,4R)-3-fluoro-4-(4-methoxyphenyl)-1-(2-methylpyridin-4-yl)pyrrolidin-3-yl]carbonyl}-4- (methoxymethyl)pyrrolidin-3-yl]-5-(trifluoromethyl)phenyl}piperidine-4-carboxylic acid;
1-{2-[(3S,4S)-4-fluoro-1-{[(3R,4R)-3-fluoro-1-(trans-4-methoxycyclohexyl)-4-(4-methoxyphenyl)pyrrolidin-3-yl]carbonyl}-4-(methoxymethyl)pyrrolidin-3-yl]-5-(trifluoromethyl)phenyl}piperidine-4-carboxylic acid;
1-{5-chloro-2-[(3S,4R)-1-{[(3R,4R)-3-fluoro-1-(trans-4-methoxycyclohexyl)-4-(4-methoxyphenyl)pyrrolidin-3-yl]carbonyl}-4-(methoxymethyl)pyrrolidin-3-yl]phenyl}piperidine-4-carboxylic acid;
1-{2-[(3S,4R)-4-(cyanomethyl)-1-{[(3R,4R)-1-cyclopentyl-3-fluoro-4-(4-methoxyphenyl)pyrrolidin-3-yl]carbonyl}pyrrolidin-3-yl]-5-(trifluoromethyl)phenyl}piperidine-4-carboxylic acid;
1-{2-[(3S,4R)-1-{[(3R,4R)-3-fluoro-1-(trans-4-methoxycyclohexyl)-4-(4-methoxyphenyl)pyrrolidin-3-yl]carbonyl}-4- (methoxymethyl)pyrrolidin-3-yl]-5-(trifluoromethyl)phenyl}piperidine-4-carboxylic acid;
1-[2-(1-{[(3R,4R)-3-fluoro-1-(trans-4-methoxycyclohexyl)-4-(4-methoxyphenyl)pyrrolidin-3-yl]carbonyl}piperidin-4-yl)-5- (trifluoromethyl)phenyl]piperidine-4-carboxylic acid;
1-{2-[(3S,4R)-1-{[(3R,4R)-1-cyclopentyl-3-fluoro-4-(4-methoxyphenyl)pyrrolidin-3-yl]carbonyl}-4-(methoxymethyl)pyrrolidin-3- yl]-5-(trifluoromethyl)phenyl}piperidine-4-carboxylic acid; and
1-{2-[(3S)-1-{[(3R,4R)-3-fluoro-1-(trans-4-methoxycyclohexyl)-4-(4-methoxyphenyl)pyrrolidin-3-yl]carbonyl}pyrrolidin-3-yl]-5-(trifluoromethyl)phenyl}piperidine-4-carboxylic acid;
   or a pharmaceutically acceptable salt thereof.

Preferably, the invention is directed to a pyrrolidine compound or pharmaceutically acceptable salt selected from the group consisting of:
1-{2-[(3S,5S)-1-{[(3R,4R)-1-(trans-4-ethoxycyclohexyl)-3-methoxy-4-(4-methoxyphenyl)pyrrolidin-3-yl]carbonyl}-5- (ethoxymethyl)pyrrolidin-3-yl]-5-trifluoromethyl)phenyl}piperidine-4-carboxylic (acid;
1-{5-fluoro-2-[(3S,5S)-1-{[(3R,4R)-3-fluoro-1-(trans-4-methoxycyclohexyl)-4-(4-methoxyphenyl)pyrrolidin-3-yl]carbonyl}-5-(methoxymethyl)pyrrolidin-3-yl]phenyl}piperidine-4-carboxylic acid;
1-{2-[(3S,5S)-1-{[(3R,4R)-1-(trans-4-ethoxycyclohexyl)-3-methoxy-4-(4-methoxyphenyl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,5S)-1-{[(3R,4R)-1-(trans-4-cyanocyclohexyl)-3-fluoro-4-(4-methoxyphenyl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,4S)-1-{[(3R,4R)-1-(trans-4-ethoxycyclohexyl)-3-methoxy-4-(4-methoxyphenyl)pyrrolidin-3-yl]carbonyl}-4-fluoro-4-(methoxymethyl)pyrrolidin-3-yl]-5-(trifluoromethyl)phenyl}piperidine-4-carboxylic acid;
1-{2-[(3S,4R)-1-{[(3R,4R)-1-(trans-4-ethoxycyclohexyl)-3-methoxy-4-(4-methoxyphenyl)pyrrolidin-3-yl]carbonyl}-4- (methoxymethyl)pyrrolidin-3-yl]-5-(trifluoromethyl)phenyl}piperidine-4-carboxylic acid;
1-{2-[(3S,5S)-1-{[(3R,4R)-1-cyclohexyl-3-fluoro-4-(4-methoxyphenyl)pyrrolidin-3-yl]carbonyl}-5-(methoxymethyl)pyrrolidin-3- yl]-5-(trifluoromethyl)phenyl}piperidine-4-carboxylic acid;
1-{2-[(3S,5S)-1-{[(3R,4R)-3-fluoro-1-(trans-4-methoxycyclohexyl)-4-(4-methoxyphenyl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,5S)-1-{[(3R,4R)-1-cyclopentyl-3-fluoro-4-(4-methoxyphenyl)pyrrolidin-3-yl]carbonyl}-5-(methoxymethyl)pyrrolidin-3- yl]-5-(trifluoromethyl)phenyl}piperidine-4-carboxylic acid;
1-{2-[(3S,4S)-4-fluoro-1-{[(3R,4R)-3-fluoro-1-(trans-4-methoxycyclohexyl)-4-(4-methoxyphenyl)pyrrolidin-3-yl]carbonyl}-4-(methoxymethyl)pyrrolidin-3-yl]-5-(trifluoromethyl)phenyl}piperidine-4-carboxylic acid;
1-{5-chloro-2-[(3S,4R)-1-{[(3R,4R)-3-fluoro-1-(trans-4-methoxycyclohexyl)-4-(4-methoxyphenyl)pyrrolidin-3-yl]carbonyl}-4-(methoxymethyl)pyrrolidin-3-yl]phenyl}piperidine-4-carboxylic acid;
1-{2-[(3S,4R)-4-(cyanomethyl)-1-{[(3R,4R)-1-cyclopentyl-3-fluoro-4-(4-methoxyphenyl)pyrrolidin-3-yl]carbonyl}pyrrolidin-3-yl]-5-(trifluoromethyl)phenyl}piperidine-4-carboxylic acid;
1-{2-[(3S,4R)-1-{[(3R,4R)-3-fluoro-1-(trans-4-methoxycyclohexyl)-4-(4-methoxyphenyl)pyrrolidin-3-yl]carbonyl}-4- (methoxymethyl)pyrrolidin-3-yl]-5-(trifluoromethyl)phenyl}piperidine-4-carboxylic acid;
1-[2-(1-{[(3R,4R)-3-fluoro-1-(trans-4-methoxycyclohexyl)-4-(4-methoxyphenyl)pyrrolidin-3-yl]carbonyl}piperidin-4-yl)-5- (trifluoromethyl)phenyl]piperidine-4-carboxylic acid;
1-{2-[(3S,4R)-1-{[(3R,4R)-1-cyclopentyl-3-fluoro-4-(4-methoxyphenyl)pyrrolidin-3-yl]carbonyl}-4-(methoxymethyl)pyrrolidin-3- yl]-5-(trifluoromethyl)phenyl}piperidine-4-carboxylic acid; and
1-{2-[(3S)-1-{[(3R,4R)-3-fluoro-1-(trans-4-methoxycyclohexyl)-4-(4-methoxyphenyl)pyrrolidin-3-yl]carbonyl}pyrrolidin-3-yl]-5-(trifluoromethyl)phenyl}piperidine-4-carboxylic acid;
   or a pharmaceutically acceptable salt thereof.

Preferably, the invention is directed to a pyrrolidine compound or pharmaceutically acceptable salt thereof of above-mentioned formula (I), wherein the compound is selected from the group consisting of:
1-{5-fluoro-2-[(3S,5S)-1-{[(3R,4R)-3-fluoro-1-(trans-4-methoxycyclohexyl)-4-(4-methoxyphenyl)pyrrolidin-3-yl]carbonyl}-5-(methoxymethyl)pyrrolidin-3-yl]phenyl}piperidine-4-carboxylic acid;
1-{2-[(3S,5S)-1-{[(3R,4R)-1-(trans-4-ethoxycyclohexyl)-3-methoxy-4-(4-methoxyphenyl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,5S)-1-{[(3R,4R)-3-fluoro-1-(trans-4-methoxycyclohexyl)-4-(4-methoxyphenyl)pyrrolidin-3-yl]carbonyl}-5- (methoxymethyl)pyrrolidin-3-yl]-5-(trifluoromethyl)phenyl}piperidine-4-carboxylic acid;
1-{2-[(3S,5S)-1-{[(3R,4R)-1-cyclopentyl-3-fluoro-4-(4-methoxyphenyl)pyrrolidin-3-yl]carbonyl}-5-(methoxymethyl)pyrrolidin-3- yl]-5-(trifluoromethyl)phenyl}piperidine-4-carboxylic acid;
1-{2-[(3S,4S)-4-fluoro-1-{[(3R,4R)-3-fluoro-1-(trans-4-methoxycyclohexyl)-4-(4-methoxyphenyl)pyrrolidin-3-yl]carbonyl}-4-(methoxymethyl)pyrrolidin-3-yl]-5-(trifluoromethyl)phenyl}piperidine-4-carboxylic acid;
1-{5-chloro-2-[(3S,4R)-1-{[(3R,4R)-3-fluoro-1-(trans-4-methoxycyclohexyl)-4-(4-methoxyphenyl)pyrrolidin-3-yl]carbonyl}-4-(methoxymethyl)pyrrolidin-3-yl]phenyl}piperidine-4-carboxylic acid;
1-{2-[(3S,4R)-1-{[(3R,4R)-3-fluoro-1-(trans-4-methoxycyclohexyl)-4-(4-methoxyphenyl)pyrrolidin-3-yl]carbonyl}-4- (methoxymethyl)pyrrolidin-3-yl]-5-trifluoromethyl)phenyl}piperidine-4-carboxylic acid; and
1-{2-[(3S,4R )-1-{[(3R,4R)-1-cyclopentyl-3-fluoro-4-(4-methoxyphenyl)pyrrolidin-3-yl]carbonyl}-4-(methoxymethyl)pyrrolidin-3- yl]-5-(trifluoromethyl)phenyl}piperidine-4-carboxylic acid;
   or a pharmaceutically acceptable salt thereof.

According to the present invention, the most preferred pyrrolidine compound is 1-{2-[(3S,4R)-1-{[(3R,4R)-1-cyclopentyl-3-fluoro-4-(4- methoxyphenyl)pyrrolidin-3-yl]carbonyl}-4-(methoxymethyl)pyrrolidin-3-yl]-5-(trifluoromethyl)phenyl}piperidine-4-carboxylic acid or a pharmaceutically acceptable salt thereof. This compound is often called dersimelagon and can be represented by the following formula (III):

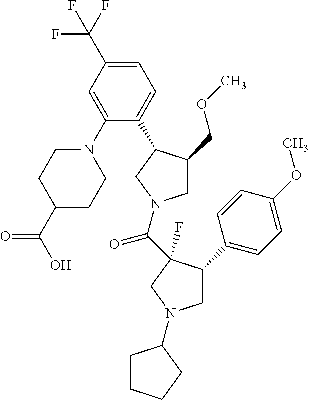

Preferred dersimelagon has CAS number 1835256-48-8 and molecular formula structure C₃₆H₄₅F₄N₃O₅ and a molecular weight of 675.753.

Preferably, the pyrrolidine compound is administered in the form of a composition, preferably a pharmaceutical composition for oral administration, such as a tablet or capsule. Preferably, the composition is a controlled release composition, resulting in longer and/or more controlled exposure of the body to the drug. Preferably, the composition preferably comprises at least 0.1 mg of the pyrrolidine compound and preferably at most 100 mg. The composition preferably comprises at least one other pharmaceutically-acceptable components can be encapsulated or incorporated in the composition. For example, the one or more pharmaceutically-acceptable component can include a fatty acid, a sugar, a salt, a water-soluble polymer such as polyethylene glycol, a protein, polysacharride, or carboxmethyl cellulose, a surfactant, a plasticizer, a high- or low-molecular-weight porosigen such as polymer or a salt or sugar, or a hydrophobic low-molecular-weight compound such as cholesterol or a wax.

### EXAMPLES

XP is a disease that can be classified in eight complementation groups XP-A to XP-G as well as XP-variant (XP-V). The cause of XP is a mutation in the genetic coding, leading to synthesis of different, dysfunctional XP proteins of different structure. While the details of the functioning of these different XP proteins in subjects suffering from XP is not fully understood, the differences have a dramatic clinical effect on the DNA repair capability, leading to high DNA damage, skin cancer and death at a young age.

Despite the high level of DNA damage occurring in XP patients, we realized that XP patients have remaining—low or very low—DNA repair capability. We further realized that such residual activity however means that the complexes and pathways in XP patients are at least partially intact. Based on these insights and despite the XP protein in XP patients having a different structure with associated dysfunctionality, we realized not only that these different XP proteins are still being recruited for complexes used in the DNA repair pathways, but also—importantly—that administration of pyrrolidine compounds according to the invention to XP patients further enhances such recruitment of factors—including the different XP protein—for these complexes. Further, we realized that pyrrolidine compounds of the invention often better associate with various allele variants of the MC1R receptor associated with DNA repair in comparison with natural alpha-MSH levels, rendering the subsequent factor recruitment and DNA repair in XP patients more effective. We also realized that exposure of XP patient to pyrrolidine compounds over longer periods enhances factor recruitment and subsequent DNA repair even further. We conclude that use of pyrrolidine compounds of the invention in XP patients leads to an improved DNA repair capability reducing the cancer risk for XP patients particularly when compared to alpha-MSH at natural levels.

The above shows that the pyrrolidine compounds are suitable and can be successfully used for DNA repair in XP patients which the following clinical trial confirms:

Subjects diagnosed with XP based on clinical symptoms and genotyping are assigned to one of the 8 XP subgroups, including at least XP-A, XP-B, XP-C, XP-E, XP-F and XP-V. Subjects can be orally administered pyrrolidine compounds according to the invention on a daily basis. Skin biopsies are taken according to the preferred method described in reference (literature reference 1): Dreze M, Calkins A S, Ga'licza J, Echelman D J, Schnorenberg M R, et al. (2014) “Monitoring Repair of UV-Induced 6-4-Photoproducts with a Purified DDB2 Protein Complex”. PLoS ONE 9(1): e85896. doi:10.1371/journal.pone.0085896. Biopsies are taken before (comparison), during and after periods of exposure to the most preferred pyrrolidine compound of the invention. The biopsies are used to determine the concentration of photoproducts and dimers, such as 6,4 CPD and 8-oxoguanine. A preferred method is described in reference (literature reference 2) McCready S. (2014), “An Immunoassay for Measuring Repair of UV Photoproducts”. In: Keohavong P., Grant S. (eds) Molecular Toxicology Protocols. Methods in Molecular Biology (Methods and Protocols), vol 1105. Humana Press, Totowa, NJ.

The methods are useful for measuring repair in total genomic DNA, and are thought to be sufficiently sensitive to measure repair of damage induced by light and UV radiation. Repair of genomic material will be confirmed after administration of the preferred pyrrolidine compound (compared to before administration) and show the positive and unexpected beneficial effects on DNA repair in subjects suffering from XP.

### EXPERIMENTAL IN-VITRO SET-UP AND RESULTS WITH DERSIMELAGON

### Measurement of UV-Induced Apoptosis by Annexin Va Staining

Melanocytes were maintained for the duration of the experiment in medium lacking bovine pituitary extract (BPE) to determine the survival effect of dersimelagon (synthesized with reference to US2017190697) vs control in the absence of the anti-apoptotic effect of TPA. Melanocytes were treated with the different concentrations of the pyrrolidine compound for 4 days prior to, and 24 h after exposure to a UV dose (105 mJ/cm²), then stained for Annexin Va staining. The data were collected as percent increase above control, with triplicate dishes included in each group. Effect of the pyrrolidine compound vs control on the UV-induced apoptosis were determined in melanocyte strains. Floating as well as attached cells were harvested and stained for Annexin V, and analyzed by flow cytometry. Dersimelagon had beneficial anti-apoptotic effects, supporting the effects of pyrrolidine compounds of the invention in treating human subjects suffering from XP.

1. Pyrrolidine compound for use in the treatment of a human subject suffering from Xeroderma Pigmentosum (XP), wherein the pyrrolidine compound is represented by formula [I]: 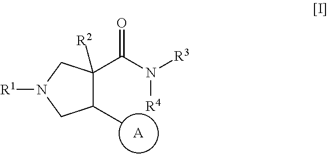
wherein
ring A represents an optionally substituted aryl group or an optionally substituted heteroaryl group;
R¹ represents an optionally substituted alkyl group, an optionally substituted cycloalkyl group, an optionally substituted aliphatic heterocyclic group, an optionally substituted aryl group that may be partially hydrogenated, an optionally substituted heteroaryl group, or an optionally substituted carbamoyl group;
R² represents a halogen atom, an alkyl group, or an optionally substituted alkoxy group;
R³ is an alkyl group substituted with an optionally substituted aryl group, or an alkyl group substituted with an optionally substituted heteroaryl group and R⁴ is a hydrogen atom or an alkyl group; or R³ and R⁴ are terminally attached to each other, and together with the nitrogen atom to which they are attached, form an optionally substituted nitrogen-containing aliphatic heterocyclic ring that may partially contain a double bond;
or a pharmaceutically acceptable salt thereof.

2. Compound for use according to claim 1, wherein the compound is used in enhancing DNA repair in the treatment of a human subject suffering from Xeroderma Pigmentosum (XP).

3. Compound for use according any of claims 1-2, wherein the compound is a 3,3-di-substituted pyrrolidine compounds with 2 substituents in the 3-position of pyrrolidine and wherein the compound preferably has substituents in the 1-, 3-, and 4-positions.

4. Compound for use according to any of claims 1 to 3, wherein the pyrrolidine compound is 1-{2-[(3S,4R)-1-{[(3R,4R)-1-cyclopentyl-3-fluoro-4-(4- methoxyphenyl)pyrrolidin-3-yl]carbonyl}-4-(methoxymethyl)pyrrolidin-3-yl]-5-(trifluoromethyl)phenyl}piperidine-4-carboxylic acid or a pharmaceutically acceptable salt thereof.

5. Compound for use according to any of claims 1 to 4, wherein the compound is administered orally.

6. Compound for use according to any of claims 1-5, wherein the compound is administered daily.

7. Compound for use according to any of claims 1 to 6, wherein the compound is administered at least 3 times consecutively to the human subject.

8. Compound for use according to any of claims 1 to 7, wherein XP is selected from complementation group A (XP-A), complementation group B (XP-B), complementation group C (XP-C), complementation group D (XP-D), complementation group E (XP-E), complementation group F (XP-F), complementation group G (XP-G), and variant type (XP-V).

9. Compound for use according to any of claims 1 to 7, wherein the compound is used for treatment of complementation group C (XP-C).

10. Method of treating Xeroderma Pigmentosum (XP) by administering a pyrrolidine compound.

11. Method according to claim 10, wherein the compound is administered to a human subject with an interval between subsequent administrations of the pyrrolidine compound is between 1 to 15 days.

12. Use of a pyrrolidine compound for the manufacture of a medicament for the treatment of Xeroderma Pigmentosum.
